(19) United States
(12) Patent Application Publication
(10) Pub. No.: US 2014/0135235 A1
(43) Pub. Date: May 15, 2014

(54) GLYCAN AND GLYCOPEPTIDE CAPTURE AND RELEASE USING REVERSIBLE HYDRAZONE-BASED METHOD

(71) Applicants: Hui Zhang, Ellicott City, MD (US); Shuang Yang, Ellicott City, MD (US)

(72) Inventors: Hui Zhang, Ellicott City, MD (US); Shuang Yang, Ellicott City, MD (US)

(21) Appl. No.: 14/124,288

(22) PCT Filed: Jun. 6, 2012
(86) PCT No.: PCT/US2012/041050

§ 371 Date: Jan. 16, 2014

Related U.S. Application Data
(60) Provisional application No. 61/493,694, filed on Jun. 6, 2011.

(51) Int. Cl. G01N 33/68

(52) U.S. Cl. CPC G01N 33/6842
USPC 506/11; 436/94; 435/272; 506/23

(57) ABSTRACT

Highly specific and novel methods for reversible hydrazone solid-phase extraction (rHSPE) are provided for glycan or glycopeptide isolation from proteins, peptides, and other contaminants for glycan and glycopeptide analysis. Glycans or glycopeptides in complex mixtures can be conjugated onto solid support or affinity or chemical tags via reversible hydrazone bond. The conjugation methods of the present invention are chemically specific and provide unique means for the removal of other non-glycan containing molecules in the complex sample before the glycans or glycopeptides are hydrolyzed and recovered for analysis. The hydrazone formation and hydrolysis of the novel methods allows for the analysis of glycans and glycopeptides. The hydrazide coating on the solid-phase surfaces are useful for surface glycan capture and on target glycan analysis. Uses of the information generated by the inventive methods for diagnosis and treatment are also disclosed.

### REFERENCE TO RELATED APPLICATIONS

This application claims the benefit of U.S. Provisional Patent Application No. 61/493,694, filed on Jun. 6, 2011, which is hereby incorporated by reference for all purposes as if fully set forth herein.

### STATEMENT OF GOVERNMENTAL INTEREST

This invention was made with U.S. government support under grant nos. HHSN268201000032C and UO1CA152813. The U.S. government has certain rights in the invention.

### BACKGROUND OF THE INVENTION

Protein glycosylation has been considered as one of the most significant protein modifications. It has been widely recognized that glycosylation is associated with disease progression, such as cancer, heart failure, and other congenital disorders. The investigation of glycoproteins and their associated glycans is the key to understanding glycoprotein functions in biological pathways and disease development as well as biomarker discovery. To this end, the inventors previously developed the solid-phase extraction of glycopeptides (SPEG) for capture of glycosylated peptides, which has been widely applied to both quantitative analysis of glycoproteins and identification of glycosylation sites. In this method, glycosylated peptides from digested glycoproteins are captured by using hydrazide beads after glycans on glycopeptides are oxidized. Following the removal of non-glycosylated peptides, these glycosylated peptides are then enzymatically released from the solid support for mass spectrometry (MS) analysis. Using this method, thousands of new N-linked glycosylation sites have been identified. However, the glycans are removed from glycopeptides during the capture processes and their structures are not identified.

Several methods have been developed for glycan analysis. Typically, glycans are first released from glycoproteins or glycopeptides by enzymes such as Peptide: N-Glycosidase F (PNGase F) for N-linked glycans or by chemicals reactions, like β-elimination for O-linked glycans. Upon the release, glycans are desalted and purified from enzymes, chemicals, and their concatenate peptides for mass spectrometry analysis. Although glycans are purified by separating them from peptides and other non-glycan molecules by using a variety of methods such as affinity column, reverse-phase high-performance liquid chromatography, capillary electrophoresis, hydrophilic interaction chromatography, or multidimensional separations, the major obstacle for these methods is their incapability to separate glycans or glycopeptides from other species, especially from the non-glycosylated peptides. In terms of glycan purification, the graphite guard column is a widely used medium for glycan purification, mostly for the removal of salts and small molecules. However, the graphite column separates glycans and other molecules in the complex samples based on hydrophobicity; the column will also isolate the nonspecific hydrophilic species and the low molecular weight of peptides in the glycan fraction. As a result, the yield and specificity of glycans recovered from complex glycoprotein samples remain low. Therefore, there still exists an unmet need to develop analytical methods which can specifically isolate glycans or glycopeptides from complex mixtures.

### SUMMARY OF THE INVENTION

Using hydrazide coated superparamagnetic silica particles, a novel and highly specific approach called “reversible hydrazone solid phase extraction” (rHSPE) was developed to isolate glycans or glycopeptides from the complex mixture by reversible reaction to hydrazide beads. The reducing ends of glycans or oxidized glycans or glycopeptides containing aldehyde groups react to the hydrazide on the bead surface, conjugating glycans or glycopeptides on beads. After washing the beads, the glycans or glycopeptides were released from beads using acids and analyzed by mass spectrometry. The novel methods of the present invention provide the means to isolate glycans or glycopeptides from other components in complex mixture for glycomics and glycoproteomics analysis, and present potential uses for clinical applications.

In accordance with an embodiment, the present invention provides a method of isolating glycans in a biological sample comprising: a) obtaining a biological sample comprising glycoproteins; b) denaturing the sample of a) to release the glycans from the glycoproteins; c) conjugating the glycans from b) to a solid support; d) removing the non-glycan species from the sample from c); e) hydrolyzing the glycans from the solid support of c); and f) isolating the glycans released from the solid support of c).

In accordance with another embodiment the present invention provides a method of identifying glycans in a biological sample comprising: a) obtaining a biological sample comprising glycoproteins; b) denaturing the sample of a) to release the glycans from the glycoproteins; c) conjugating the glycans from b) to a solid support; d) removing the non-glycan species from the sample; e) hydrolyzing the glycans from the solid support of c); f) isolating the glycans released from the solid support of c); and g) analyzing the glycans of f).

In accordance with an embodiment the present invention provides a method of isolating glycans and glycopeptides in a biological sample comprising: a) obtaining a biological sample comprising glycoproteins; b) oxidizing the glycans of glycopeptides; c) conjugating the glycopeptides to a solid support; d) collecting non-glycopeptides and optionally analyzing them; e) releasing glycopeptides (both N- and O-glycopeptides) from the solid support by hydrolysis; or optionally f) releasing formerly N-glycopeptides by PNGase F; g) releasing O-glycopeptides and N-glycans from the solid support via hydrolysis of hydrazone bonds; h) isolating N-glycans via affinity separation or optionally by rHSPE; i) isolating O-glycopeptides; or optionally j) releasing O-glycans from O-glycopeptides and isolating O-glycans and peptides.

In accordance with another embodiment the present invention provides a method of identifying glycans and glycopeptides in a biological sample comprising: a) obtaining a biological sample comprising glycoproteins; b) oxidizing the glycans of glycopeptides; c) conjugating the glycopeptides to a solid support; d) collecting non-glycopeptides and optionally analyzing them; e) releasing glycopeptides (both N- and O-glycopeptides) from the solid support by hydrolysis and analyzed; or optionally f) releasing formerly N-glycopeptides by PNGase F and analyzing them; g) releasing O-glycopeptides and N-glycans from the solid support via hydrolysis of hydrazone bonds and analyzed; h) isolating N-glycans via affinity separation or optionally by rHSPE; i) isolating O-glycopeptides and analyzing them; or optionally j) releasing O-glycans from O-glycopeptides and isolating O-glycans and peptides and analyzed.

In accordance with a further embodiment the present invention provides a method of generating a library comprising a glycans and glycopeptides using the above identified methods.

In accordance with still another embodiment, the present invention provides a method of detection and/or diagnosis of a disease or condition in a subject comprising generating a glycan or glycopeptide profile from a sample from the subject using the above identified methods, and comparing the glycan profile of the subject to a standard or normal profile and determining whether the subject has the disease or condition.

### BRIEF DESCRIPTION OF THE DRAWINGS

FIG. 1 is a schematic diagram of glycan capture by reversible hydrazone solid-phase extraction (rHSPE) for glycan analysis. Glycans are first conjugated; non-glycan molecules are removed by washing, and glycans are hydrolyzed from the solid phase in acidic buffer.

FIG. 2 is another schematic diagram of comprehensive glycoprotein analysis by solid-phase extraction of glycans and glycopeptides via reversible hydrazone formation and hydrolysis. Glycoproteins are digested to peptides and glycopeptides (i); glycans on glycopeptides are oxidized and conjugated to hydrazide beads (ii); non-glycopeptides are collected and optionally analyzed (iii); while glycopeptides remain on beads (iv); Glycopeptides (both N- and O-glycopeptides) are released from beads by hydrolysis and analyzed (v); alternatively, formerly N-glycopeptides are released by PNGase F and analyzed (SPEG) (vi); while O-glycopeptides and N-glycans are still immobilized on beads (vii); O-glycopeptides and N-glycans are then released from beads via hydrolysis of hydrazone bonds (viii); N-glycans are isolated via affinity separation or rHSPE and analyzed (ix); O-glycopeptides are isolated and analyzed (x); or alternatively, O-glycans are released from O-glycopeptides and analyzed separately for O-glycans and peptides.

FIG. 3 is also a schematic diagram of capture and release glycans using hydrazide on solid-phase beads. Aldehyde is first formed from cyclic form under catalysis of aniline. At an acidic condition (pH 3-5), the hydrazide on beads conjugates to glycan by microwave irradiation and formation of hydrazone between glycan and the bead. The uncoupled components in the sample mixture are washed away from the beads. At a stronger acidic condition (pH 1-2), the hydrazone is hydrolyzed to release glycan from the bead. The beads can be replaced with slides coated with compounds with amino groups.

FIG. 4 depicts the reversible hydrazone formation of Man-9 to ADH in solution. The conjugation was in 10 mM acetate buffer (pH 5.0) and 10 mM aniline using microwave radiation. The molecular weight of Man-9/ADH conjugation is 2062.63 Da (mono-Na⁺) (middle spectrum). Without conjugation prior to reaction, the mass spectrum (bottom) only showed Man-9 (mono-Na⁺) (1906.68 Da); after reaction with ADH, the mass was 2062.63 Da (mono-Na⁺) (middle); after hydrolysis by heating at 60° C./1 h in 10% formic acid, Man-9 was recovered, indicating hydrolysis of hydrazone.

FIG. 5 depicts Man-9 conjugation in acetate buffer (pH 5.0) (a) and PBS buffer (pH 7.4) (b). In PBS buffer, the conversion of Man-9 to Man-9-ADH was less than 15% based on the peak intensities; in acetate buffer, more than 90% Man-9 was conjugated to ADH.

FIG. 6 shows the effect of aniline on Man-9 conjugation to hydrazide. The conjugation with (1) and without (b) 10 mM aniline was compared.

FIG. 7 depicts the effect of amount of magnetic beads on glycan recovery. All four DPs were recovered using 5 mL of beads (a) and 1 mL of beads solution (b); while no DPs were detectable using 100 μL, of beads (c).

FIG. 8 shows glycan conjugation and hydrolysis on hydrazide beads from a mixture of glycans (DPs) and peptides (NT, neurotensin and AG, angiotensin I). The mixed sample includes DP5 (851.25 Da), DP6 (1013.28 Da), DP7 (1175.31 Da), angiotensin I (mono-H⁺) (1296.68 Da), and neurotensin (mono-H⁺) (1672.77 Da) (peptide peaks other than angiotensin I and neurotensin were detected in mixed samples, which were due to the impurity of sample purchased). After conjugation, washing, and hydrolysis, glycans (DP5, DP6, and DP7) were detected by MALDI-MS. Angiotensin I and neurotensin were detected in washing solution but not in solution hydrolyzed from the bead surface.

FIG. 9 depicts glycan isolation from human serum with the addition of standard glycans and peptides by hydrazide beads. The bottom spectrum was the sample mixture without glycan isolation. The top spectrum was isolated glycans from hydrazide beads after conjugation, washing, and hydrolysis.

FIG. 10 shows a table with a portion of the glycans isolated from a human sample.

### DETAILED DESCRIPTION OF THE INVENTION

Methods are provided herein which are directed to improved methods of analyzing carbohydrates. As used herein, the term “carbohydrate” is intended to include any of a class of aldehyde or ketone derivatives of polyhydric alcohols. Therefore, carbohydrates include starches, celluloses, gums and saccharides. Although, for illustration, the term “saccharide” or “glycan” is used below, this is not intended to be limiting. It is intended that the methods provided herein can be directed to any carbohydrate, and the use of a specific carbohydrate is not meant to be limiting to that carbohydrate only.

As used herein, the term “saccharide” refers to a polymer comprising one or more monosaccharide groups. Saccharides, therefore, include mono-, di-, tri- and polysaccharides (or glycans). Glycans can be branched or branched. Glycans can be found covalently linked to non-saccharide moieties, such as lipids or proteins (as a glycoconjugate). These covalent conjugates include glycoproteins, glycopeptides, peptidoglycans, proteoglycans, glycolipids and lipopolysaccharides. The use of any one of these terms also is not intended to be limiting as the description is provided for illustrative purposes. In addition to the glycans being found as part of a glycoconjugate, the glycans can also be in free form (i.e., separate from and not associated with another moiety). The use of the term peptide is not intended to be limiting. The methods provided herein are also intended to include proteins where “peptide” is recited.

In accordance with an embodiment, the present invention provides a method of isolating glycans in a biological sample comprising: a) obtaining a biological sample comprising glycoproteins; b) denaturing the sample of a) to release the glycans from the glycoproteins; c) conjugating the glycans from b) to a solid support; d) removing the non-glycan species from the sample; e) hydrolyzing the glycans from the solid support of c); and f) isolating the isolated glycans released from the solid support of c).

In another embodiment the present invention provides a method of identifying glycans in a biological sample comprising: a) obtaining a biological sample comprising glycoproteins; b) denaturing the sample of a) to release the glycans from the glycoproteins; c) conjugating the glycans from b) to a solid support; d) removing the non-glycan species from the sample; e) hydrolyzing the glycans from the solid support of c); f) isolating the isolated glycans released from the solid support of c); and g) analyzing the glycans of f).

In accordance with another embodiment of the present invention, it will be understood that the term “biological sample” or “biological fluid” includes, but is not limited to, any quantity of a substance from a living or formerly living subject. Such substances include, but are not limited to, blood, serum, plasma, urine, cells, organs, tissues, bone, bone marrow, lymph, lymph nodes, synovial tissue, CSF, chondrocytes, synovial macrophages, endothelial cells, and skin. In a preferred embodiment, the fluid is blood or serum.

As used herein, the term “subject” refers to any mammal, including, but not limited to, mammals of the order Rodentia, such as mice and hamsters, and mammals of the order Logomorpha, such as rabbits. It is preferred that the mammals are from the order Carnivora, including Felines (cats) and Canines (dogs). It is more preferred that the mammals are from the order Artiodactyla, including Bovines (cows) and Swines (pigs) or of the order Perssodactyla, including Equines (horses). It is most preferred that the mammals are of the order Primates, Ceboids, or Simoids (monkeys) or of the order Anthropoids (humans and apes). An especially preferred mammal is the human.

In accordance with an embodiment the present invention provides a method of isolating glycans and glycopeptides in a biological sample comprising: a) obtaining a biological sample comprising glycoproteins; b) oxidizing the glycans of glycopeptides; c) conjugating the glycopeptides to a solid support; d) collecting non-glycopeptides and optionally analyzing them; e) releasing glycopeptides (both N- and O-glycopeptides) from the solid support by hydrolysis; or optionally f) releasing formerly N-glycopeptides by PNGase F; g) releasing O-glycopeptides and N-glycans from the solid support via hydrolysis of hydrazone bonds; h) isolating N-glycans via affinity separation or optionally by rHSPE; i) isolating O-glycopeptides; or optionally j) releasing O-glycans from O-glycopeptides and isolating O-glycans and peptides.

In accordance with another embodiment the present invention provides a method of identifying glycans and glycopeptides in a biological sample comprising: a) obtaining a biological sample comprising glycoproteins; b) oxidizing the glycans of glycopeptides; c) conjugating the glycopeptides to a solid support; d) collecting non-glycopeptides and optionally analyzing them; e) releasing glycopeptides (both N- and O-glycopeptides) from the solid support by hydrolysis and analyzed; or optionally f) releasing formerly N-glycopeptides by PNGase F and analyzing them; g) releasing O-glycopeptides and N-glycans from the solid support via hydrolysis of hydrazone bonds and analyzed; h) isolating N-glycans via affinity separation or optionally by rHSPE; i) isolating O-glycopeptides and analyzing them; or optionally j) releasing O-glycans from O-glycopeptides and isolating O-glycans and peptides and analyzed.

It will be understood by those of skill in the art that the denaturation of the glycoproteins in the sample, in the inventive methods, can be accomplished using any means known in the art. In addition to heating, denaturation agents and proteolysis may also be used. A “denaturing agent” is an agent that alters the structure of a molecule, such as a protein. Denaturing agents, therefore, include agents that cause a molecule, such as a protein to unfold. Denaturing can be accomplished, for instance, with heat, with heat denaturation in the presence of β-mercaptoethanol and/or SDS, by reduction followed by carboxymethylation (or alkylation), etc. Reduction can be accomplished with reducing agent, such as, dithiothreitol (DTT). Carboxymethylation or alkylation can be accomplished with, for example, iodoacetic acid or iodoacetamide. Denaturation can, for example, be accomplished by reducing with DTT, β-mercaptoethanol or tri(2-carboxyethyl)phosphine (TCEP) followed by carboxymethylation with iodoacetic acid. When the glycoconjugate sample is a sample of a body fluid, such as serum, the denaturation can be accomplished with EndoF. The glycoconjugates can also be denatured with denaturing agents, such as detergent, urea or guanidium hydrochloride.

In accordance with an embodiment, the methods of analyzing glycans of the present invention includes cleaving the glycans from the glycoconjugates using any chemical or enzymatic methods or combinations thereof that are known in the art. An example of a chemical method for cleaving glycans from glycoconjugates is hydrazinolysis or alkali borohydrate. Enyzmatic methods include methods that are specific to N- or O-linked sugars. These enzymatic methods include the use of Endoglycosidase H (Endo H), Endoglycosidase F (EndoF), N-Glycanase F (PNGaseF) or combinations thereof. In some preferred embodiments, PNGaseF is used when the release of N-glycans is desired. When PNGaseF is used for glycan release the proteins is, for example, first unfolded prior to the use of the enzyme. The unfolding of the protein can be accomplished with any of the denaturing agents provided above.

In accordance with an embodiment of the above methods of the present invention the denaturing of the sample in a) comprises: i) heating the sample for a sufficient period of time; ii) incubating the sample from i) with a proteolytic enzyme for a period of time; and iii) adding a sufficient amount of PNGase F to the sample of ii) to release the glycans from the peptide fragments.

It is understood by those of skill in the art that the proteolytic enzyme used in the inventive methods can be any enzyme capable of cleaving peptide bonds. Examples of proteolytic enzymes useful in the inventive methods include trypsin, chymotrypsin, papain, and pepsin.

After protein denaturation and/or digestion, the (If using Endo H, the peptide portion still containing carbohydrate) peptides and protein fragments can be removed by washing or use of various column based methods known in the art. Alternatively, the peptides and protein fragments can be collected and separately analyzed using known methods.

In accordance with an embodiment, the methods of the present invention include conjugating the free glycans in sample or the released glycans from glycoconjugates to a solid support. In an embodiment, conjugation of the free glycans or the released glycans of the sample in b) comprises: i) adding at least a portion of the sample from b) to a solid support comprising superparamagnetic hydrazide nanoparticles; ii) mixing the mixture of i); and iii) incubating the mixture of ii) for a sufficient time at a temperature of between 40-60° C. This allows the released glycans to form hydrazone bonds to the hydrazide moieties on the nanoparticles. In another embodiment, the hydrazide moieties could be ligated or otherwise chemically bound to any known solid support.

In accordance with a further embodiment, the conjugation of the released glycans to the solid support is performed in the absence or presence of a catalyst. It will be understood by those of skill in the art that catalysts suitable for use with the methods of the present invention will include those compounds that can act as a Schiff-base intermediate in the reaction of the free reducing ends of the glycans with the hydrazide moieties on the solid support. In the inventive methods, the catalyst can be added to the mixture of the released glycans and solid support. In accordance with an embodiment, the catalyst used in the inventive methods is aniline.

The solid substrate used to bind the glycans and glycopeptides in the inventive methods may be a material that may be modified to contain discrete individual sites appropriate for the attachment or association of the glycans and is amenable to at least one detection method. Representative examples of substrates include glass and modified or functionalized glass, plastics (including acrylics, polystyrene and copolymers of styrene and other materials, polypropylene, polyethylene, polybutylene, polyurethanes, Teflon, etc.), polysaccharides, nylon or nitrocellulose, resins, silica or silica-based materials including silicon and modified silicon, carbon, metals, inorganic glasses and plastics.

In accordance with an embodiment, the solid substrate is a porous monolith, in which hydrazides are functionalized onto a porous monolith surface. When the glycan mixture is made to flow through the porous monolith, the glycans are immobilized on its surface while other non-glycans are removed in the flow-through. It will be understood by those of skill in the art that this can be implemented in a capillary or a microfluidic platform

In an alternative embodiment, other supports, such as slides, for example can be used as the solid support. This is particularly useful for glycan analysis with spatial information and can be applied to glycan imaging of tissues. In yet another embodiment tags can also be used in place of solid supports, using well known ligands such as biotin-hydrazide or azido-hydrazide, that can be used to conjugate the glycans in solution and then are subsequently captured using the tags. This solution capture embodiment is particularly useful to capture glycans in vivo.

In accordance with an embodiment, the solid substrate used in the methods of the present invention is a nanoparticle having a superparamagnetic ferrous core. The nanoparticles of the present invention also comprise a silica composition which can be derivatized. In one or more embodiments, the silica coated nanoparticles have hydrazine or other amine group containing moieties attached thereto.

The mixture containing the glycans and glycopeptides are incubated for a period of time with the solid support to allow the glycans with aldehyde to conjugate to the hydrazide moieties on the support to form a hydrazone bond. In some embodiments, the heating took between about 10 minutes to about 30 minutes, at temperature between about 40° C.-60° C. While any type of heating can be used, in a preferred embodiment, heating is performed using a microwave oven. In an embodiment, the glycans and the hydrazide moieties on the support to form a hydrazone bond on a nanoparticle having a superparamagnetic ferrous core. The magnetic nature of the particles allows easier separation from the non-glycan components in the digested mixture and subsequent washings.

After the removal of the non-conjugated components, the glycans or glycopeptides (both N- and O-glycopeptides) can be released from beads by hydrolysis and analyzed. In accordance with an embodiment, the hydrolysis of the hydrazone bonds is accomplished by lowering the pH of the solution to <3. In some embodiments the range of pH is between about pH 1 to about pH 3, preferably about pH 2. While it will be understood that any acid solution can be used to accomplish this, such as, for example trifluoroacetic acid (<1% v/v), 0.01 M HCl, or 0.005 M H₂SO₄. In accordance with an embodiment, 10% v/v formic acid is suitable for this use.

In accordance with an embodiment, the step of analyzing the glycans includes, in certain embodiments, analyzing the glycans with a mass spectrometric method, an electrophoretic method, NMR, a chromatographic method or a combination thereof. In a further embodiment, the mass spectrometric method is LC-MS and LC-MS/MS using LC-Orbitrap, LC-FTMS, LC-LTQ, MALDI-MS including but not limited to MALDI-TOF, MALDI-TOF/TOF, MALDI-qTOF, and MALDI-QIT. Preferably, the mass spectrometric method is a quantitative MALDI-MS or LC-MS using optimized conditions. In still another embodiment, the electrophoretic method is CE-LIF. In yet another embodiment, methods such as capillary gel electrophoresis or capillary zone electrophoresis can be used with the inventive methods.

In other embodiments, includes quantifying the glycans using calibration curves of known glycan standards.

In yet another embodiment, the methods of the present invention include a method for diagnostic or prognostic purposes.

In a further embodiment, the methods of the present invention include a method for assessing the purity of the sample.

In some embodiments, the methods are methods of diagnosis and the pattern is associated with a diseased state. In one preferred embodiment, the pattern associated with a diseased state is a pattern associated with cancer, such as prostate cancer, melanoma, bladder cancer, breast cancer, lymphoma, ovarian cancer, lung cancer, colorectal cancer or head and neck cancer. In other preferred embodiments, the pattern associated with a diseased state is a pattern associated with an immunological disorder; a neurodegenerative disease, such as a transmissible spongiform encephalopathy, Alzheimer's disease or neuropathy; inflammation; rheumatoid arthritis; cystic fibrosis; or an infection, preferably viral or bacterial infection. In other embodiments, the method is a method of monitoring prognosis and the known pattern is associated with the prognosis of a disease. In yet another embodiment, the method is a method of monitoring drug treatment and the known pattern is associated with the drug treatment. In particular, the methods (e.g., analysis of glycome profiles) are used for the selection of population-oriented drug treatments and/or in prospective studies for selection of dosing, for activity monitoring and/or for determining efficacy endpoints.

Methods of analyzing glycans of glycoconjugates can also include cleaving the glycans from glycoconjugates using any chemical or enzymatic methods or combinations thereof that are known in the art. An example of a chemical method for cleaving glycans from glycoconjugates is hydrazinolysis or alkali borohydrate. Enyzmatic methods include methods that are specific to N- or O-linked sugars. These enzymatic methods include the use of Endoglycosidase H (Endo H), Endoglycosidase F (EndoF), N-Glycanase F (PNGaseF) or combinations thereof. In some preferred embodiments, PNGaseF is used when the release of N-glycans is desired. When PNGaseF is used for glycan release the proteins is, for example, first unfolded prior to the use of the enzyme. The unfolding of the protein can be accomplished with any of the denaturing agents provided above.

After the release of the glycan from the protein core, or when the glycans were already in free form (not part of a glycoconjugate), the sample can be purified, for instance, by precipitating the proteins with ethanol and removing the supernatant containing the glycans. Other experimental methods for removing the proteins, detergent (from a denaturing step) and salts include any methods known in the art. These methods include dialysis, chromatographic methods, etc. In one example, the purification is accomplished with a porous graphite column. In some preferred embodiments, everything but the glycans is removed from the sample. Samples can also be purified with commercially available resins and cartridges for clean-up after chemical cleavage or enzymatic digestion used to separate glycans from protein. Such resins and cartridges include ion exchange resins and purification columns, such as GlycoClean H, S, and R cartridges. Preferably, in some embodiments GlycoClean H is used for purification.

Purification can also include the removal of high abundance proteins, such as the removal of albumin and/or antibodies, from a sample containing glycans. In some methods the purification can also include the removal of unglycosylated molecules, such as unglycosylated proteins. Removal of high abundance proteins can be a desirable step for some methods, such as some high-throughput methods described elsewhere herein. In some embodiments of the methods provided, abundant proteins, such as albumin or antibodies, can be removed from the samples prior to the final composition analysis.

In other embodiments, the glycans can be modified to improve ionization of the glycans, particularly when MALDI-MS is used for analysis. Such modifications include permethylation. Another method to increase glycan ionization is to conjugate the glycan to a hydrophobic chemical (such as AA, AB labeling) for MS or liquid chromatographic detection. Examples of the methods are described further in the Examples below. In other embodiments, spot methods can be employed to improve signal intensity.

Any analytic method for analyzing the glycans so as to characterize them can be performed on any sample of glycans, such analytic methods include those described herein. As used herein, to “characterize” a glycan or other molecule means to obtain data that can be used to determine its identity, structure, composition or quantity. When the term is used in reference to a glycoconjugate, it can also include determining the glycosylation sites, the glycosylation site occupancy, the identity, structure, composition or quantity of the glycan and/or non-saccharide moiety of the glycoconjugate as well as the identity and quantity of the specific glycoform. These methods include, for example, mass spectrometry, NMR (e.g., 2D-NMR), electrophoresis and chromatographic methods. Examples of mass spectrometric methods include FAB-MS, LC-MS, LC-MS/MS, MALDI-MS, MALDI-MS/MS, etc. NMR methods can include, for example, COSY, TOCSY, NOESY. Electrophoresis can include, for example, CE-LIF, CGE, CZE, COSY, TOCSY, NOESY. Electrophoresis can include, for example, CE-LIF.

The library consists of free or labeled glycoconjugates and fragments of the glycoconjugates, the fragments being the non-saccharide portions of the glycoconjugates. In one example, a library is generated from a sample, by isolating the glycoconjugates or free glycans or by cleaving the backbone of the glycoconjugates in the sample. The glycans or glycoconjugates can then be removed from the sample. The libraries so produced can be analyzed with the methods provided herein. The libraries can also be used as a standard once characterized and methods of using such libraries are also provided.

In one embodiment, the inventive methods include a method of analyzing a sample with glycoconjugates includes isolating free forms of glycans or glycoconjugate, or cleaving the glycoconjugates by enzymatically or chemically removing the glycans from the glycoconjugates and mixing the sample with a standard. The sample mixed with the standard can then be analyzed. In one embodiment, the amounts of the glycoconjugates and non-saccharide moieties of the sample and standard are compared. In one aspect of the invention the standards are also provided.

Prior to analysis of the sample, the sample can also be degraded with a chemical or enzymatic method to cleave the glycans from any glycoconjugates in the sample. Examples of enzymatic methods are provided above and include, for example, the use of PNGase F, endoglycosydase H and endoglycosydase F or combinations thereof. Chemical methods have also been described above and include hydrazinolisis, alkali borohydrate or beta-elimination.

After chemical or enzymatic degradation the sample can then be performed in some embodiments. Purification methods were also provided above. Examples of particular purification methods include using solid phase extraction cartridges, such as graphitized carbon columns and C-18 columns.

As stated above, the glycosylation of a protein may be indicative of a normal or a disease state. Therefore, methods are provided for diagnostic purposes based on the analysis of the glycosylation of a protein or set of proteins, such as the total glycome. The methods provided herein can be used for the diagnosis of any disease or condition that is caused or results in changes in a particular protein glycosylation or pattern of glycosylation. These patterns can then be compared to “normal” and/or “diseased” patterns to develop a diagnosis, and treatment for a subject. For example, the methods provided can be used in the diagnosis of cancer, inflammatory disease, benign prostatic hyperplasia (BPH), etc.

The diagnosis can be carried out in a person with or thought to have a disease or condition. The diagnosis can also be carried out in a person thought to be at risk for a disease or condition. “A person at risk” is one that has either a genetic predisposition to have the disease or condition or is one that has been exposed to a factor that could increase his/her risk of developing the disease or condition.

Detection of cancers at an early stage is crucial for its efficient treatment. Despite advances in diagnostic technologies, many cases of cancer are not diagnosed and treated until the malignant cells have invaded the surrounding tissue or metastasized throughout the body. Although current diagnostic approaches have significantly contributed to the detection of cancer, they still present problems in sensitivity and specificity.

In accordance with one or more embodiments of the present invention, it will be understood that the types of cancer diagnosis which may be made, using the methods provided herein, is not necessarily limited. For purposes herein, the cancer can be any cancer. As used herein, the term “cancer” is meant any malignant growth or tumor caused by abnormal and uncontrolled cell division that may spread to other parts of the body through the lymphatic system or the blood stream.

The cancer can be a metastatic cancer or a non-metastatic (e.g., localized) cancer. As used herein, the term “metastatic cancer” refers to a cancer in which cells of the cancer have metastasized, e.g., the cancer is characterized by metastasis of a cancer cells. The metastasis can be regional metastasis or distant metastasis, as described herein.

The terms “treat,” and “prevent” as well as words stemming therefrom, as used herein, do not necessarily imply 100% or complete treatment or prevention. Rather, there are varying degrees of treatment or prevention of which one of ordinary skill in the art recognizes as having a potential benefit or therapeutic effect. In this respect, the inventive methods can provide any amount of any level of diagnosis, staging, screening, or other patient management, including treatment or prevention of cancer in a mammal. Furthermore, the treatment or prevention provided by the inventive method can include treatment or prevention of one or more conditions or symptoms of the disease, e.g., cancer, being treated or prevented. Also, for purposes herein, “prevention” can encompass delaying the onset of the disease, or a symptom or condition thereof.

In accordance with an embodiment, the present invention provides a use of a glycan profile prepared using the method disclosed herein to diagnose a disease or condition in a subject, comprising comparing the glycan profile from a subject to a glycan profile from a normal sample, or diseased sample, and determining whether the sample of the subject has the disease or condition.

In accordance with the inventive methods, the terms “cancers” or “tumors” also include but are not limited to adrenal gland cancer, biliary tract cancer; bladder cancer, brain cancer; breast cancer; cervical cancer; choriocarcinoma; colon cancer; endometrial cancer; esophageal cancer; extrahepatic bile duct cancer; gastric cancer; head and neck cancer; intraepithelial neoplasms; kidney cancer; leukemia; lymphomas; liver cancer; lung cancer (e.g. small cell and non-small cell); melanoma; multiple myeloma; neuroblastomas; oral cancer; ovarian cancer; pancreas cancer; prostate cancer; rectal cancer; sarcomas; skin cancer; small intestine cancer; testicular cancer; thyroid cancer; uterine cancer; urethral cancer and renal cancer, as well as other carcinomas and sarcomas.

### EXAMPLES

Materials and Reagents. All chemicals and reagents were purchased from Sigma-Aldrich (St. Louis, Mo.) unless specified. Matrix assisted laser desorption/ionization (MALDI) matrix (2,5-dihydroxybenzoic acid (DHB, >99.0% purity, 30 mg/mL)) was freshly prepared in 50% methanol and 0.1% TFA solution in an amber glass vial (Tubing Vials, Fisher Scientific, Pittsburgh, Pa.). In-house synthesized hydrazide coated superparamagnetic silica particles (15.2 μm in diameter) were used for the glycan capture using the method outlined in Anal. Chem., 80:1228-1234 (2008), and incorporated by reference herein. Maltotetraose (DP4), maltopentose (DP5), maltohexanose (DP6), and maltoheptaose (DP7) were from Sigma-Aldrich.

Peptide and Glycan Preparation. Two standard peptides, angiotensin I human acetate salt hydrate (AG, 1296.68 Da, mono-H⁺, 0.39 mg) and neurotensin (NT, 1672.91 Da, mono-H⁺, 0.50 mg), were dissolved in 300 μL of high-pressure liquid chromatography (HPLC) grade water. Standard glycans, Man-9 ((Man)₉(GlcNAc)₂, 1883.67 Da, 20 μg), maltopentose (DP5, 851.26 Da, mono-Na⁺, 7.2 mg), maltohexanose (DP6, 1013.31 Da, mono-Na⁺, 8.6 mg), and maltoheptaose (DP7, 1175.37 Da, mono-Na⁺, 10 mg) were dissolved in HPLC grade water, forming 0.21 mM of Man-9 glycan and 10 mM of the four DP mixture. A volume of 5 μL of serum was dissolved into 200 μL of 0.4 mM ammonium bicarbonate buffer (pH 8.0-8.3), followed by denaturation at 100° C. for 5 minutes. After cooling the sample to room temperature, trypsin at 500 μL of 1 μg/μL was added into the serum solution and incubated at 37° C. for 16 hours. The digested sample was heated at 100° C. for 5 minutes to deactivate trypsin before adding 10 μL of PNGase F (500 U/μL, New England BioLabs Inc., Ipswich, Mass.). The sample was then incubated at 37° C. for 16 hours, dried in a Speed-Vac, and resuspended in HPLC water. Before analyzing peptides and glycans from the digested solution, the sample was purified using a graphite column (Grace Davison Discovery Sciences, Milwaukee, Wis.) to remove the salts and other small molecules.

Glycan-Bead Conjugation. One mL of magnetic beads was placed in 1.5 mL of polypropylene snap-cap microcentrifuge tube (Fisher Scientific, Pittsburgh, Pa.). The magnetic beads were pre-conditioned using 1 mL of 85:15 solution of methanol:acetate buffer (150 mL of acetate buffer in 850 mL of HPLC grade of methanol), repeated two more times. The supernatant was then removed after the beads had stuck on the tube sidewall using magnetic particle separator (Invitrogen Corporation, Carlsbad, Calif.). Five μL of glycan and peptide mixture was added to the beads containing 100 μL of acetate buffer and methanol. The aniline was added as a catalyst with a final concentration 10 mM to perform conjugation of glycan reducing ends to hydrazide beads. The sample was mixed with beads in microcentrifuge tube over vortex mixer (VM-3000, VWR International, Radnor, Pa.) and the mixture was placed in a microwave oven (EMS-820; Electron Microscopy Sciences, Hatfield, Pa.) to react for 20 minutes at 50° C. with microwave oven power at 50%. To avoid over-heating sample-beads solution, 1-2 minute interval was set at every 5 minutes microwave irradiation.

Glycan Release. The conjugated glycans on beads were rinsed using 1 mL of 50 mM ammonium bicarbonate (pH 8.0-8.3). Nonconjugated samples (including peptides) were washed away after five times of rinsing. Formic acid (200 μL, 10%) was added into cleaned beads, which were placed in a 60° C. oven and incubated for 60 minutes. After cooling to room temperature, supernatant was collected using magnetic separator and beads were rinsed two more times by 200 μL of 5% acetic acid solution. The supernatant was cleaned using a Carbograph Extract-Clean LC column (Grace Davison Discovery Sciences, Milwaukee, Wis.), which was activated by 0.1% TFA in 50% acetonitrile (3 mL, 2×) and 0.1% TFA in HPLC grade water (3 mL, 3×) sequentially. Samples were eluted by 50% acetonitrile with 0.1% formic acid (800 μL, 2×). After drying in Savant Speed-Vac (Thermo Scientific, Asheville, N.C.), HPLC grade of solution (15 μL of 0.1% TFA in 50% methanol/DI) was added to resuspend the samples.

Alternatively, formerly N-glycopeptides are released by PNGase F and analyzed by SPEG, while O-glycopeptides and N-glycans are still immobilized on beads. This is followed by releasing the O-glycopeptides and N-glycans from the beads via hydrolysis of hydrazone bonds with 10% formic acid. The N-glycans are isolated via affinity separation or rHSPE and analyzed and O-glycopeptides are isolated and analyzed

MALDI-MS. The 384-well spot of μFocus MALDI plate (Hudson Surface Technology, Fort Lee, N.J.) was used. This plate was treated for sample concentration on surface to improve detection sensitivity. The sample of 1.5 μL was added onto MALDI plate and mixed with 1.5 μL of DHB matrix (30 mg/mL DHB). The MALDI-MS was performed with Shimadzu AXIMA Resonance (Shimdazu, Columbia, Md.) with positive mode.

### Example 1

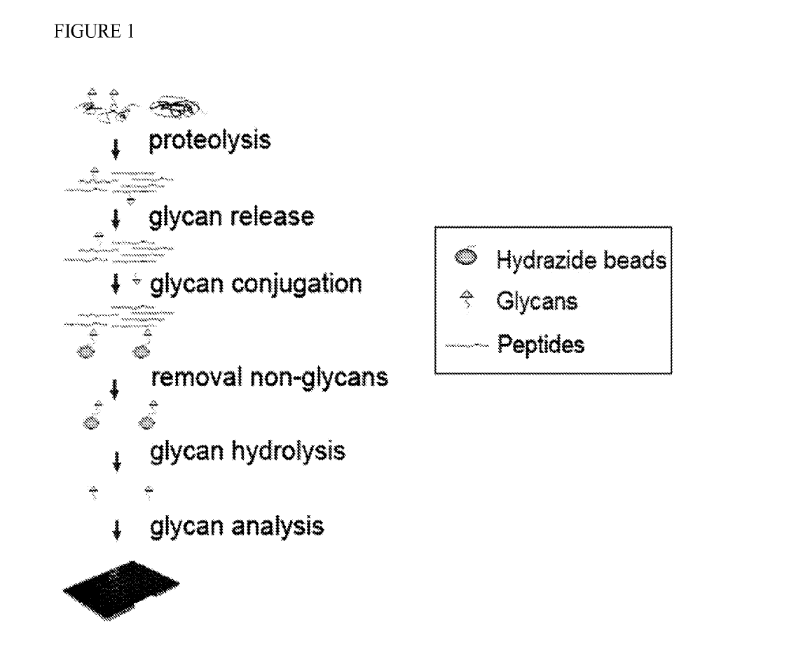

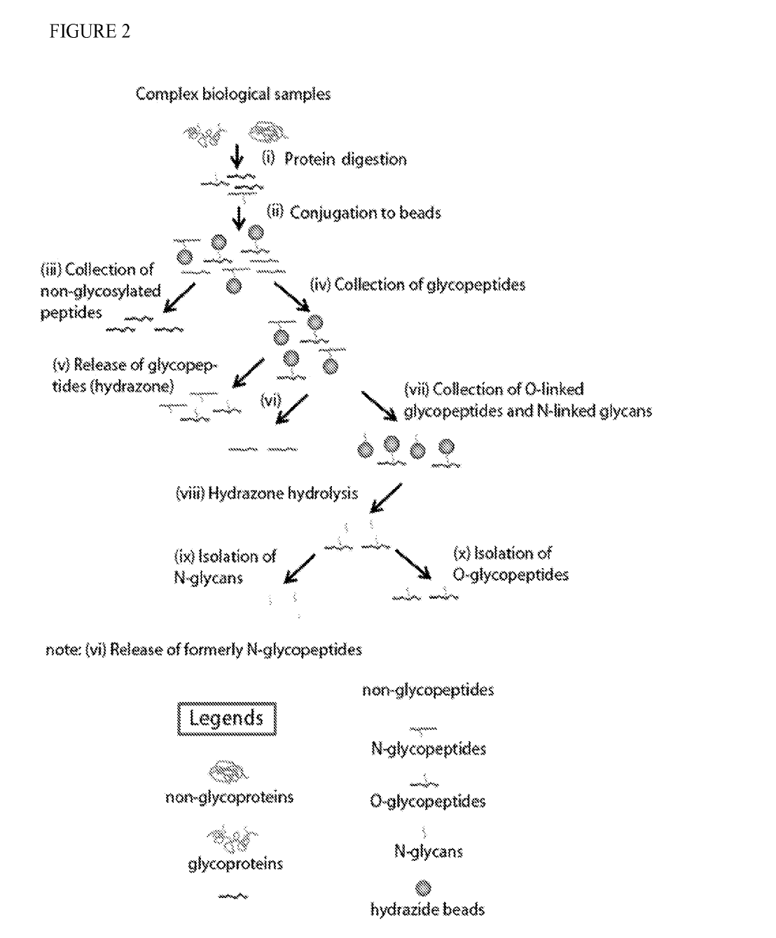

Glycan Capture Using Reversible Hydrazone Solid-Phase Extraction (rHSPE). In order to analyze intact glycans from glycoproteins, a novel method of glycan isolation was developed using reversible hydrazone solid-phase extraction (rHSPE). The steps are described in the following (FIG. 1): (1) glycan release from glycoproteins, glycans are released from denatured glycopeptides; (2) glycan conjugation to solid support, the glycans in complex mixture are then conjugated on magnetic hydrazide beads at reducing ends; (3) removal nonglycans, beads are rinsed and washed to remove other species in sample mixture; (4) glycans hydrolysis from solid support, bead-glycan conjugation are incubated in an acidic condition (pH<3.0) and glycans are hydrolyzed from beads; (5) glycan analysis, the released glycans are collected and analyzed.

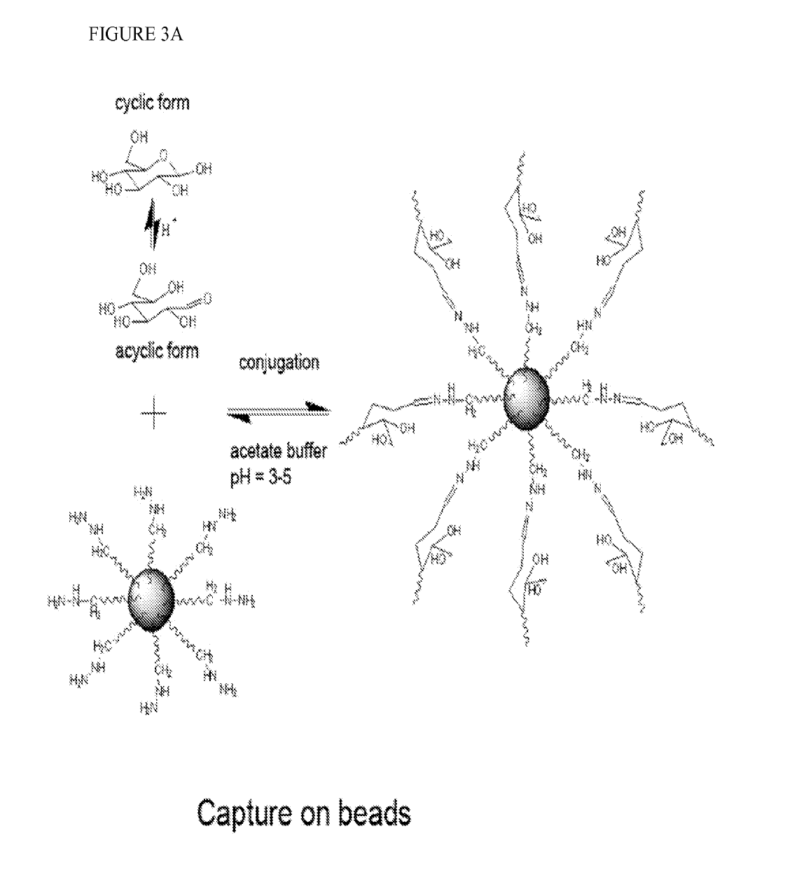

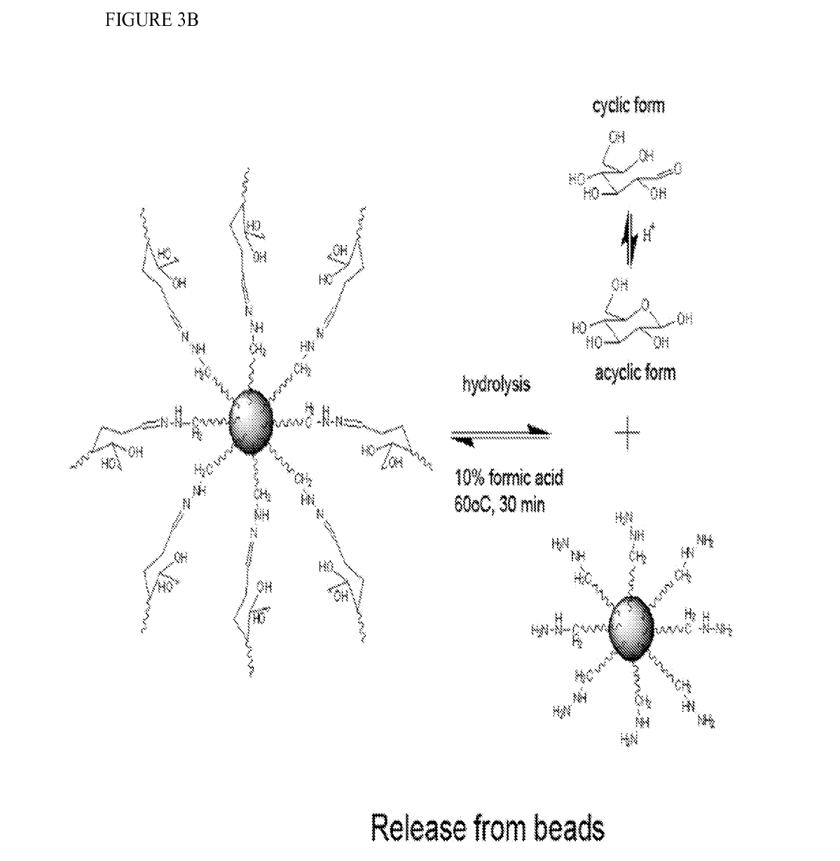

Each released glycan possesses a reducing end, which can form an aldehyde group in an acidic condition (pH 3.0-5.5). Because of their dominant ring structure, the nucleophilic reagent is used to favorably attack the ring structure, eventually forming an acyclic structure with the aldehyde group. The aldehyde then reacts with hydrazide on the solid support through the formation of hydrazone bond. The glycan-hydrazide conjugation is hydrolyzed when the conjugated glycans are incubated at 60° C. in 10% formic acid solution as shown in FIG. 3.

### Example 2

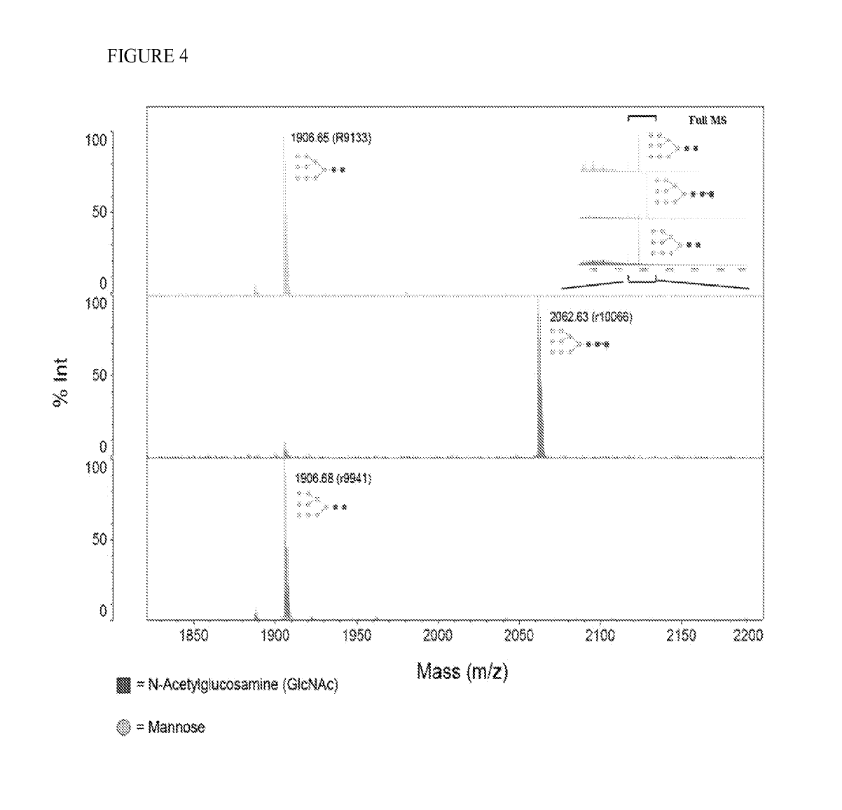

Glycan Hydrazide Conjugation and Hydrolysis. To determine the conjugation and releasing for glycans to hydrazide, we analyzed glycan conjugation and hydrolysis in solution with adipic acid dihydrazide (ADH) (FIG. 4). We tested glycans before and after conjugation as well as before and after hydrolysis by MALDI-MS. Although MALDI-MS is not the best method to precisely quantify the glycan, we used the peak intensity of each compound to estimate its relative abundance.

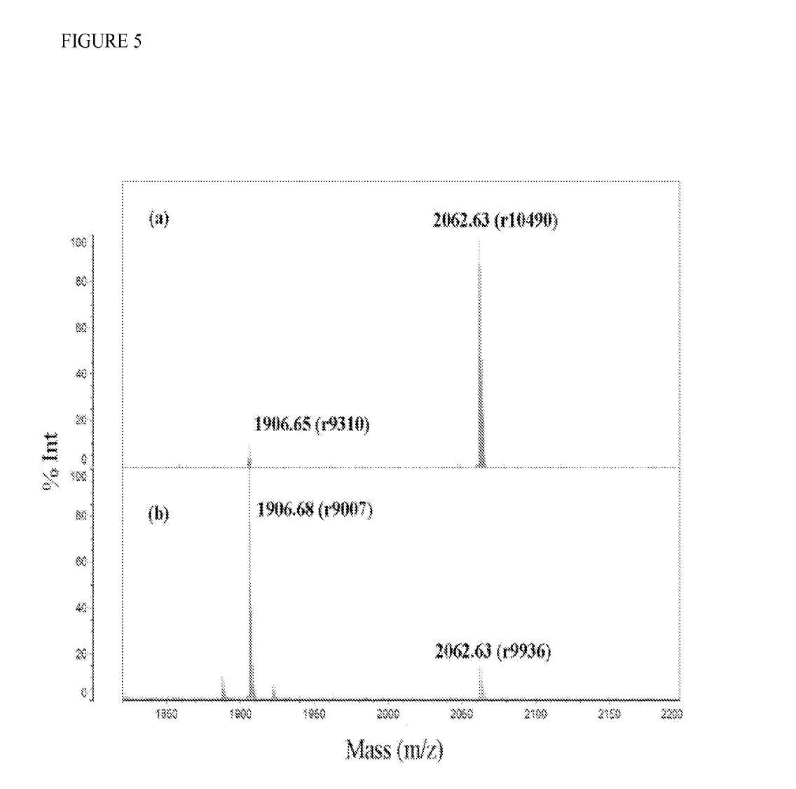

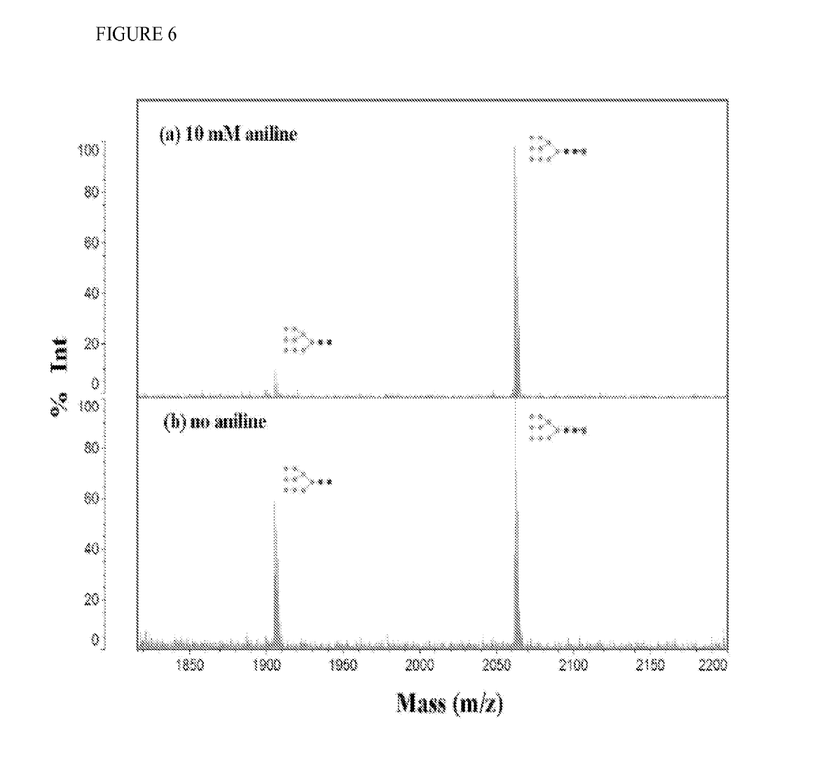

To find right pH condition for glycan conjugation, we conducted Man-9 conjugation studies at pH 7.0 and 5.0. The adipic acid dihydrazide (ADH) was dissolved in 10 mM acetate buffer (pH 5.0) and PBS buffer (pH 7.4) with the final concentration of 10 mM. Man-9 was dissolved into both buffers with the final concentration of 0.21 mM. 2 μL Man-9 was mixed with 4.2 μL ADH solution to form ADH:Man-9 of 100:1 ratio with 10 mM aniline solution. The mixed solution was heated in a microwave (EMS-820; Electron Microscopy Sciences, Hatfield, Pa.) to react for 20 minutes at 50° C. with microwave oven power at 50%. At neutral pH conditions, less than 10% conjugation was observed by estimation of the intensity ratio between glycan and conjugation product with ADH and Man-9 at a ratio of 100:1. For the reaction at pH 5.0, the conjugation was significantly improved up to approximately 90% (FIG. 5).

In this example, the conjugation also required a catalyst. For example, it was believed that formation of a Schiff-based intermediate by aniline lowered the reaction Gibbs free energy. To that end, the molar ratio of 100:1 (ADH:Man-9) was consistently used for all other cases unless specified. Aniline was purchased from Sigma-Aldrich with density 1.022 g/mL. To prepare 10 mM aniline solution, we added 0.91 μL pure aniline in 1 mL acetic buffer. 2 μL Man-9 was mixed with 4.2 μL ADH in 1 mL acetic buffer (pH 5.0) with and without 10 mM aniline solution, and incubated the mixture in microwave for 20 minutes. A high conjugation of ADH and Man-9 was observed with addition of 10 mM aniline, and the conjugation was significantly decreased without addition of aniline (FIG. 5). For glycan releasing, complete hydrolysis of the conjugated glycan-hydrazide was accomplished by adding 10% formic acid. FIG. 4 demonstrates the complete process of glycan-hydrazide conjugation and hydrolysis. In pH 5.0 acetate buffer, the mixture of Man-9, ADH, and aniline reacted via microwave irradiation (20 min, 50% power), forming conjugation Man-9-ADH (mono-Na+) (2062.63 Da) (FIG. 4, middle spectrum). The conjugated products were then mixed with 200 μL of formic acid solution (10%, vol) and heated at 60° C. for 60 min. The full MS scan was showed that the Man-9-ADH was hydrolyzed into original Man-9 (1906.65 Da) without glycan degradation (FIG. 4, insert). This reversible conjugation-hydrolysis process demonstrates the ability to use hydrazide on the solid-phase for conjugation and hydrolysis of glycans.

### Example 3

Solid-Phase Glycan Capture-Release. The conjugation-hydrolysis in solution is adapted to the solid-phase by conjugating glycans to hydrazide coated superparamagnetic silica particles. As shown in FIG. 3, the beads coated with hydrazide on the surface can use the similar principle of reversible hydrazone bond formation to isolate glycans by conjugation and hydrolysis as implemented in the solution. Because hydrazide is anchored on the beads, it has advantages over the solution to separate other components such as peptides, enzymes, and chemicals that are in solution after conjugation of glycans on hydrazide beads.

Although the chemistry of hydrazide on beads is similar to that in solution, the reaction still needs to be further adapted for hydrazide-glycan conjugation on beads due to the thermodynamic (diffusion in solution vs. absorption on surface) and surface morphology difference. For glycan conjugation to beads, 70% methanol and 30% acetate buffer (pH 5.0) was used because it was observed that beads easily settled down in acetate buffer alone while they were uniformly suspended in the 70% methanol and acetic buffer to increase the reaction hydrodynamics. The other conjugation-hydrolysis conditions determined from in-solution used the glycan conjugation-release on beads.

A key step for high glycan recovery is to complete the conjugation reaction and keep the hydrazone stable during the washing process. In solid phase capture, the reaction involves two equilibrium steps, between the acyclic form [symbol] cyclic form and between glycan-hydrazide [symbol] hydrazone. Any factor that favors the formation of acyclic and hydrazone can significantly contribute to the higher yield on conjugations.

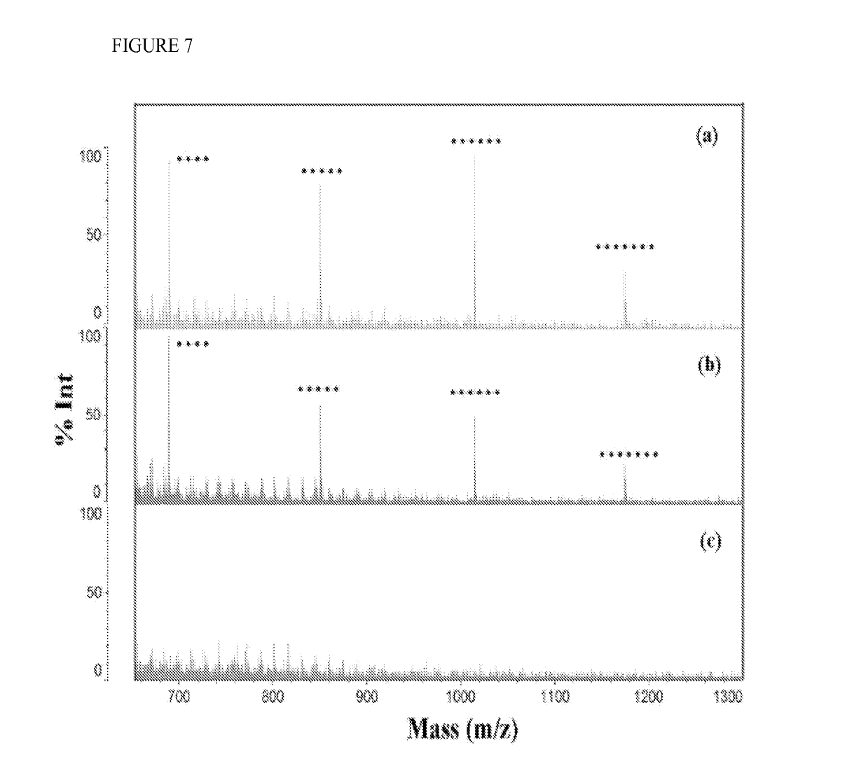

It was expected that an increased amount of beads was favorable for conjugation since it increases a higher amount of hydrazide to react with. To determine the optimal amount of hydrazide beads used to conjugate glycans, different amounts of hydrazide-beads, 0.1, 1, and 5 mL, were prepared for the glycan capture. The amount of magnetic beads is dependent on the bead geometry, surface hydrazide chemistry, and hydrazide density. In practice, it is necessary to characterize the specific beads from different vendors to determine the appropriate amount of beads. In the present invention, the beads were synthesized in the lab. The same amounts of the samples (DP4, DP5, DP6, DP7; 10 mM, 10 μL) were kept. The beads were first conditioned by 1 mL methanol-acetic buffer for 5 minutes, and liquid was removed, repeating one more time. The DPs were added into 100 μL methanol-acetic buffer with 10 mM aniline and mixed with different amount of beads. The sample and beads were mixed on vortex mixer (VM-3000, VWR International, Radnor, Pa.) and placed in a microwave oven (EMS-820; Electron Microscopy Sciences, Hatfield, Pa.) to react for 20 minutes at 50° C. with microwave oven power at 50%. To avoid over-heating bead solution, 1-2 minute interval was set at every 5 minutes microwave irradiation. There was sufficient recovery using 1 mL of original bead solution and there were no significant increase in recovery using 5 mL of bead solution. No DPs were recovered using beads less than 100 μL, suggesting that 1 mL of bead solutions is optimal to conjugate glycans. The results showed that 1 mL of beads has a similar recovered yield compared to 5 mL of beads for capture on glycans (FIG. 7).

### Example 4

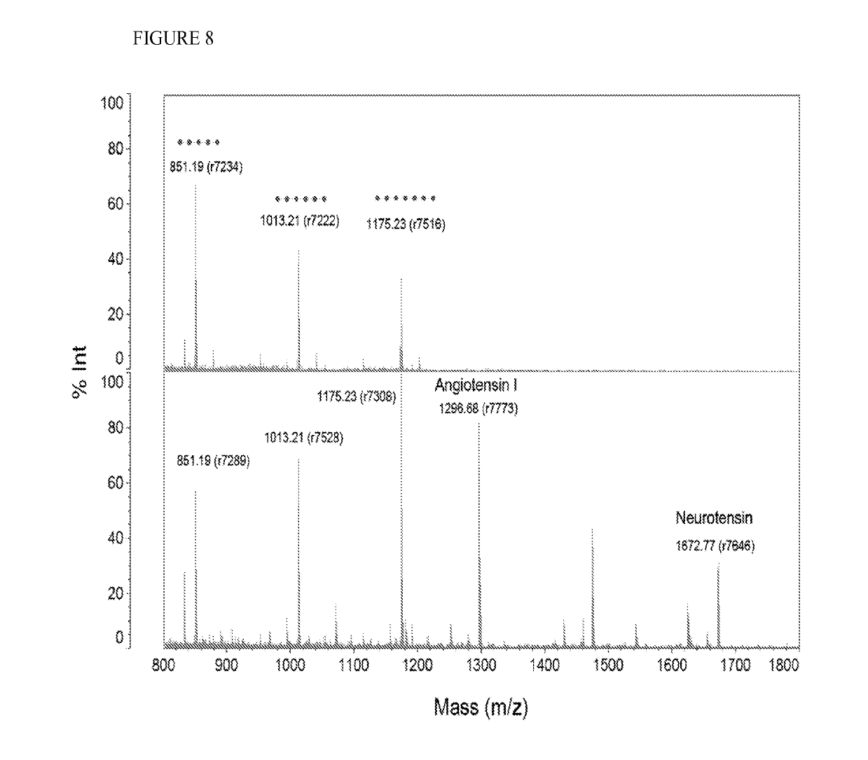

To determine the conjugation and hydrolysis conditions, a series of mixtures were constructed including standard glycans, standard peptides, mixture of standard glycans and peptides, and mixture of complex glycans and peptides from a complex biological sample (human serum) (Table 1). A volume of 5 μL was taken from 20 μL of sample mixture (Table 1). Each sample was mixed with beads in methanol-acetic buffer solution (85:15, vol/vol). The supernatant in each step was collected and dried in a Speed-Vac at 37° C. Beads were then immersed in 200 μL, 10% formic acid for 60 minutes at 60° C. The results showed that both standard peptides were removed from the glycan-peptide mixture after hydrazide-bead conjugation, washing, and hydrolysis (FIG. 8, top spectrum) even though peptides were present in the original mixture (FIG. 8, bottom spectrum). Glycans such as DP5, DP6, and DP7 were recovered in the hydrolysis solution (top spectrum). The results demonstrated the ability of bead-hydrazide for glycan isolation and analysis.

TABLE 1
Glycan and Peptide Mixtures Used for Hydrazide-Bead Conjugation
and Hydrolysis

| | | | | | | Methanol/ | |
|---|---|---|---|---|---|---|---|
| | Conc | Vol | | Conc | Vol | acetic acid | Total vol |
| Samples | (mM) | (μl) | Peptides | (mM) | (μl) | (μl) | (μl) |
| DP | 10 | 10 | NT-AG | 0.1 | 0 | 10 | 20 |
| DP | 10 | 0 | NT-AG | 0.1 | 2 | 18 | 20 |
| DP | 10 | 10 | NT-AG | 0.1 | 2 | 8 | 20 |
| DP | 10 | 10 | NT-AG | 0.1 | 2 | 3 | 20 |
| serum | | 5 | | | | | |

DPs consisted of a 10 mM mixture of DP5, DP6, and DP7. A volume of 5 μL of serum solution was used followed by digestion of trypsin and PNGase F. NT, neurotensin; AG, angiotensin I.

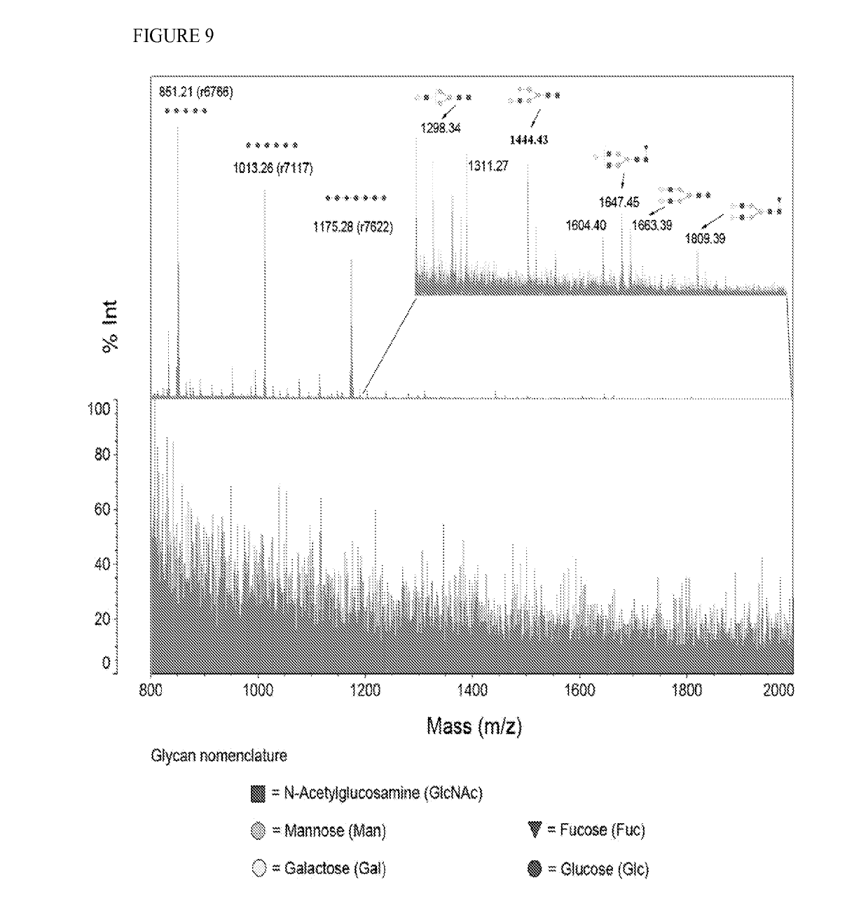

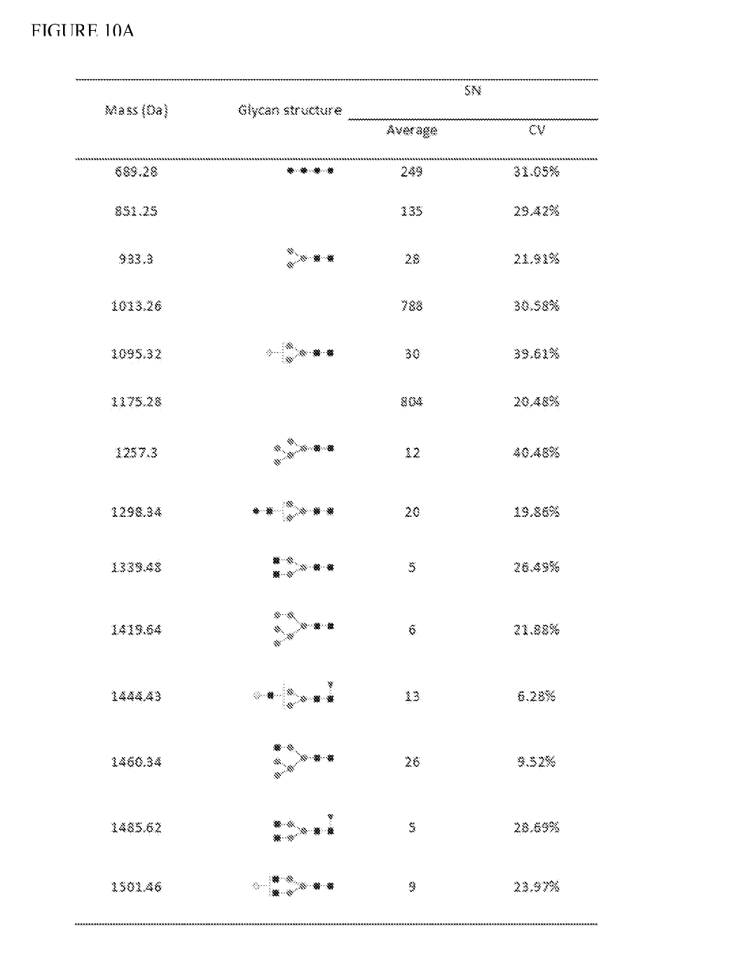

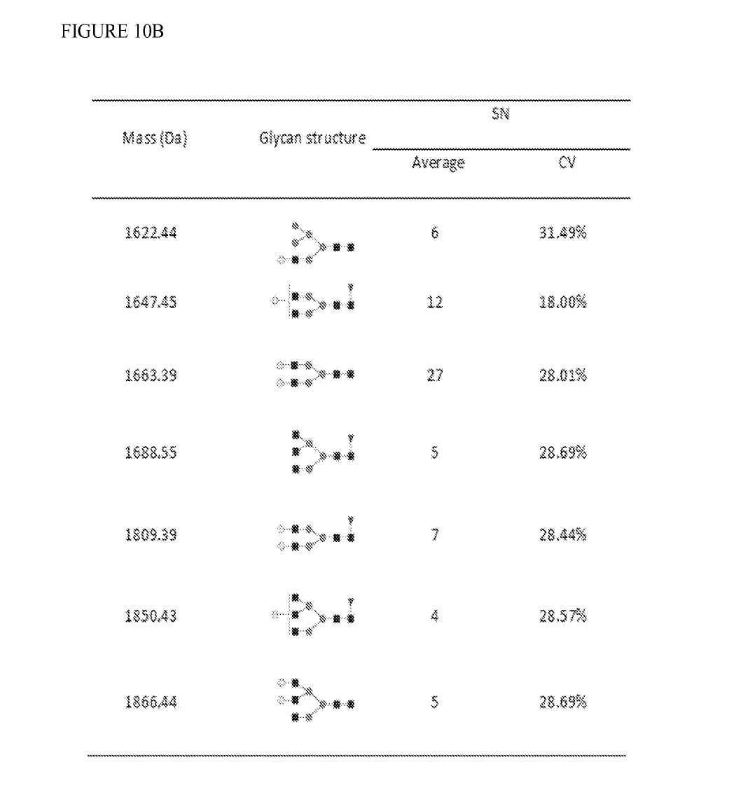

The hydrazide-beads were then applied to the analysis of a serum sample. Proteins from 20 μL of a control human serum (approximately ˜1 mg of total proteins or 10 μg of total glycans) were digested by trypsin, and glycans were released by PNGase F. The glycan and peptide mixture was dissolved into 100 μL of HPLC water. The estimated total amount of glycans was about 0.5 μg for 5 μL of prepared glycan mixture. The sample was mixed with standard glycans and peptides and then conjugated and hydrolyzed from the hydrazide beads followed by MALDI-MS analysis (FIG. 8, top). It was observed that the standard glycans, DP5, DP6, and DP7, have a strong signal after conjugation-hydrolysis from beads while the standard peptides, angiotensin I (AG) and neurotensin (NT), were not detected in the released products. Additional glycans from human serum were also detected and are shown in FIG. 8 and in the table at FIG. 10. No signal was detected in the initial sample mixture due to the high concentration of ions in the complex mixture after denaturing and trypsin and PNGase F digestion (FIG. 9, bottom). These results clearly showed that glycans were able to be isolated from the mixture using hydrazide beads.

### Example 5

The reversible hydrazone capture-release methods of the present invention were applied to the analysis of N-linked glycans from a human serum sample. A total of 17 glycans were detected by mass spectrometry after the capture-release process using the inventive methods as well four standard glycans that were spiked into human serum before glycans were isolated from the serum (data shown in Table at FIGS. 10A, 10B). It was expected that additional glycans were present in human serum samples, especially the complex glycans with terminal sialylic acids.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A method of isolating glycans in a biological sample comprising:
a) obtaining a biological sample comprising free forms of glycans or glycans from glycoproteins;
b) denaturing the sample of a) to release the glycans from the glycoproteins;
c) conjugating the free forms of glycans or glycans released from b) to a solid support;
d) removing the non-glycan species from the sample of c);
e) hydrolyzing the glycans from the solid support of c); and
f) isolating the isolated glycans released from the solid support of c).

2. The method of claim 1, further comprising g) analyzing the glycans of f).

3. A method of isolating glycans and glycopeptides in a biological sample comprising:
a) obtaining a biological sample comprising glycoproteins;
b) oxidizing the glycans of glycopeptides;
c) conjugating the glycopeptides to a solid support;
d) collecting non-glycopeptides and optionally analyzing them;
e) releasing glycans or glycopeptides (both N- and O-glycopeptides) from the solid support by hydrolysis; or optionally
f) releasing formerly N-glycopeptides by PNGase F;
g) releasing O-glycopeptides and N-glycans from the solid support via hydrolysis of hydrazone bonds;
h) isolating N-glycans via affinity separation or optionally by rHSPE; and
i) isolating O-glycopeptides; or optionally
j) releasing O-glycans from O-glycopeptides and isolating O-glycans and formerly O-linked glycopeptides.

4. A method of identifying glycans in a biological sample comprising:
a) obtaining a biological sample comprising glycoproteins;
b) oxidizing the glycans of glycopeptides;
c) conjugating the glycopeptides to a solid support;
d) collecting non-glycopeptides and optionally analyzing them;
e) releasing glycans or glycopeptides (both N- and O-glycopeptides) from the solid support by hydrolysis and analyzed; or optionally
f) releasing formerly N-glycopeptides by PNGase F and analyzing them by SPEG;
g) releasing O-glycopeptides and N-glycans from the solid support via hydrolysis of hydrazone bonds and analyzed;
h) isolating N-glycans via affinity separation or optionally by rHSPE; and
i) isolating O-glycopeptides and analyzing them; or optionally
j) releasing O-glycans from O-glycopeptides and isolation O-glycans and formerly O-linked glycopeptides and analyzed.

5. The method of claim 1, wherein the biological sample is from a subject.

6. The method of claim 1, wherein the denaturing step comprises:
i) heating the sample for a sufficient period of time;
ii) incubating the sample from i) with a proteolytic enzyme for a period of time; and
iii) adding a sufficient amount of denaturing reagents to the sample of ii) to release the glycans from the peptide fragments.

7. The method of claim 1, wherein the step of conjugating the glycans comprises:
i) adding at least a portion of the sample from b) to a solid support comprising superparamagnetic hydrazide nanoparticles;
ii) mixing the mixture of i); and
iii) incubating the mixture of ii) for a sufficient time at a temperature of between 40-60° C.

8. The method of claim 7, wherein a catalyst is added to the mixture of i).

9. The method of claim 8, wherein the catalyst is aniline.

10. The method of claim 1, wherein the step of hydrolyzing the glycans from the solid support comprises:
i) adding to the sample a sufficient amount of an acid to lower the pH of the solution to less than a pH of 3 for a sufficient period of time to allow hydrolysis of the glycans from the solid support.

11. The method of claim 2, wherein the step of analyzing is performed using an analytical method selected from the group consisting of MS, HPLC, and CE.

12. A method for preparing a library of glycans or glycopeptides from a sample comprising obtaining a sample from a subject and analyzing the glycans or glycopeptides in the sample using the methods of claim 2 to create a glycan library.

13. A method for preparing a glycan profile from a sample comprising obtaining a sample from a subject and analyzing the glycans in the sample using the methods of claim 2 to create a glycan profile.

14. A method for the diagnosis of a disease or condition in a subject comprising comparing the glycan or glycopeptide profile from a subject prepared using the method of claim 13 to a glycan profile from a normal sample or diseased sample and determining whether the sample of the subject has the disease or condition.

15. The method of claim 3, wherein the biological sample is from a subject.

16. The method of claim 4, wherein the biological sample is from a subject.

17. The method of claim 3, wherein the step of conjugating the glycans comprises:
i) adding at least a portion of the sample from b) to a solid support comprising superparamagnetic hydrazide nanoparticles;
ii) mixing the mixture of i); and
iii) incubating the mixture of ii) for a sufficient time at a temperature of between 40-60° C.

18. The method of claim 17, wherein a catalyst is added to the mixture of i).

19. The method of claim 18, wherein the catalyst is aniline.

20. The method of claim 4, wherein the step of conjugating the glycans comprises:
i) adding at least a portion of the sample from b) to a solid support comprising superparamagnetic hydrazide nanoparticles;
ii) mixing the mixture of i); and
iii) incubating the mixture of ii) for a sufficient time at a temperature of between 40-60° C.

21. The method of claim 20, wherein a catalyst is added to the mixture of i).

22. The method of claim 21, wherein the catalyst is aniline.

23. The method of claim 3, wherein the step of analyzing is performed using an analytical method selected from the group consisting of MS, HPLC, and CE.

24. The method of claim 4, wherein the step of analyzing is performed using an analytical method selected from the group consisting of MS, HPLC, and CE.
